(19) United States
(12) Patent Application Publication
(10) Pub. No.: US 2014/0194854 A1
(43) Pub. Date: Jul. 10, 2014

(54) SUBCUTANEOUS AND INTRADERMAL PATCH INFUSERS

(71) Applicant: Izrail Tsals, Newtown, PA (US)

(72) Inventor: Izrail Tsals, Newtown, PA (US)

(73) Assignee: SID TECHNOLOGIES LLC, Newtown, PA (US)

(21) Appl. No.: 14/240,902

(22) PCT Filed: Aug. 23, 2012
(86) PCT No.: PCT/US2012/052013

§ 371 Date: Feb. 26, 2014

Related U.S. Application Data
(60) Provisional application No. 61/575,749, filed on Aug. 29, 2011.

(51) Int. Cl. A61M 5/158

(52) U.S. Cl. CPC A61M 5/158
USPC 604/513; 604/170.03

(57) ABSTRACT

The device infuses a compound subcutaneously or intradermally over an extended period of time. The delivery is from a conventional prefilled syringe with a staked cannula with the device adhered to the injection site and the syringe positioned substantially parallel to the injection site. The cannula is formed by a mechanism integral to the device to provide the cannula distal section substantially perpendicular to the syringe axis. The compound delivery is based on springs or other mechanisms. The approach offers a low cost mechanical system for the infusion of high volume, viscous compounds. The activation of the patch infuser required a few simple steps by the user. A single lever is used to trigger all device functions.

### CROSS-REFERENCE TO RELATED APPLICATIONS

This PCT application claims the benefit under 35 U.S.C. §120 of U.S. Provisional Patent Application Ser. No. 61/575,749 filed Aug. 29, 2011 entitled “ID and SQ infusers” and whose entire disclosure is incorporated by reference herein.

### BACKGROUND OF THE INVENTION

The present invention relates to devices and methods for subcutaneous and intradermal injection and infusion of drugs and other compositions. Specifically subcutaneous (SQ) and intradermal (ID) rapid syringe infuser assemblies are described facilitating effective infusion from a prefilled syringe incorporated in the device.

The majority of patch pumps deliver compounds subcutaneously. This could be said about all insulin patch infusers and other infusion devices with needles. Some of the insulin patch pumps have been in production for a few years. Other devices have been or are in development.

A number of patch pumps for many compounds have been patented or are being developed by different entities. These include among others:
Abbott wearable injection device for controlled delivery of therapeutic agents, patent application U.S. Patent Publication No. 2012/0022499 (Anderson, et al.);
Beckton Dickinson Microlnfusor™ (WO 2011/075105 (Peterson, et al.));
Calibra Medical Finesse Insulin Patch-Pen;
Debiotech Jewel Pump;
Elan fully disposable MEDIPAD patch pump (U.S. Pat. No. 5,527,288 (Gross, et al.) and following patents); it delivered up to 3 ml for up to 3 days using gas drive;
Insulet Corporation (USA) OmniPod System Patch insulin pump;
Roche Medingo Solo Patch Pump (WO 2010/041260 (Yodfat, et al.));
Roche single-use electromechanical injection device (SID) developed specifically for use with the trastuzumab SC fixed-dose formulated with recombinant human hyaluronidase (U.S. Patent Publication No. 2011/0166512 (Both, et al.));
Novo Nordisk skin mountable insulin patch pump (U.S. Patent Publication No. 2011/0137255 (Nielsen, et al.));
SteadyMed Ltd. Patch pump is intended for therapeutic drugs for pain relief, diabetes and other chronic conditions and is based on proprietary ECell™ technology.
West SmartDose patch pump (Application US 2009/0093793 (Gross, et al.)); it delivers up to 3 ml SQ from a cartridge using an electromechanical drive.

These pumps have two major limitations:
the need for the user to fill the pump from a syringe, vial or merge it with a cartridge;
high cost due to a unique primary closure and the design/drive complexity.

Auto-injectors form another group of devices for SQ administration. These mechanical devices are easy to use by patients with dexterity limitations (rheumatoid arthritis, multiple sclerosis), require minimal or no training and incorporate prefilled syringes with staked cannula. The auto injectors are mechanical devices characterized by relatively low complexity and costs.

Owen Mumford, Scandinavian Health , Elcam, Rexam, West, BD and others offer auto-injectors. These injection systems are limited to the injection of up to 1 ml usually from a 1 ml long syringe. The prefilled syringe is integrated with the auto-injector in production. The compound viscosity is limited by the need to inject the content over a short time. Extended injection time is not practical when the device is manually kept in the inject position with the cannula and the elongated device perpendicular to the skin. Injection volume is limited by the ability of the subcutaneous tissue to accept rapidly injected compound without pain due to the tissue distension.

Intradermal (ID) injections are used for delivering a variety of diagnostic and treatment compositions into a patient. Substances may be injected intradermally for diagnostic testing, such as to determine a patient's immunity status against tuberculosis and the status of allergic diseases. Vaccines, drugs and other compounds may also be delivered intradermally. In many instances, intradermal delivery is preferred because it generally requires a smaller volume dose of the diagnostic or treatment compound than other delivery techniques. Furthermore the injection is less painful. An intradermal injection is made by delivering the substance into the epidermis and upper layer of the dermis. There is considerable variation in the skin thickness, both between individuals and within the same individual at different sites of the body. Generally the outer skin layer, or the epidermis, has a thickness between 500-200 microns and the dermis, the inner and thicker layer of the skin, has a thickness between 1.5-3.5 mm. Thus, for cannulae that pass beyond approximately 3.5 mm of skin depth, the type of injection is termed “subcutaneous” (SQ).

A patch pump for SQ and ID administration is detailed in U.S. Pat. No. 4,886,499 (Cirelli, et al). The patent engaging surface is relatively flat and is secured to the patient. The cannula protrudes 0.5 to 5.0 mm past the surface.

Another patch pump for ID administration is detailed in U.S. Pat. No. 5,527,288 (Gross, et al). It includes a housing with a lower surface provided with an adhesive coating for adhering the housing to the subject's skin. The protruding cannula is 0.3 to 3 mm long.

Some companies employ micro-needle systems. For example, 3M has developed and is offering infuser for rapid ID infusion of up to 1.5 ml of a compound. The delivery rate is set by an integral spring and other design parameters. The compound is contained in a cartridge and is connected to a micro-needle array by a needle engaging the cartridge during activation. The infuser is attached to the skin by an adhesive system with the delivery of up to 1.5 ml expected to take place over 3 to 40 min. “Rapid Intradermal Delivery of Liquid Formulations Using a Hollow Microstructured Array”. Scott A. Burton, Chin-Yee Ng, Kris Hansen et. al. at Springerlink.com.

The current patent application details a patch pump employing conventional prefilled syringe with a staked cannula as a primary drug closure. The term “staked” refers to the cannula being fixedly coupled to the syringe; e.g., glued or otherwise bonded to the syringe. Drug delivery SQ and ID is enabled by the device forming the cannula prior to use. The prefilled syringe is positioned at the injection site with the syringe axis primarily parallel to the skin making the patch pump ergonomically suitable for an injection over an extended period of time. The device activation including the step of the syringe cannula forming is accomplished by a single control lever. Furthermore the use of simple springs as a delivery mechanism further assures the product simplicity and low cost. Alternatively the drive mechanism could be osmotic, electromechanical or of another type.

### BRIEF SUMMARY OF THE INVENTION

The present invention relates to devices and methods for the administration of compositions into the subcutaneous and intradermal tissue layers. Patch pump device embodiments are described, according to the present invention, facilitating the use of a conventional prefilled syringe with a staked cannula. The device is merged with the conventional prefilled syringe in production. The device is activated by a control lever which forms the cannula to have its distal section primarily perpendicular to the syringe axis. The cannula insertion into the skin is also part of the device activation process achieved through same control lever as is the activation of the drug delivery mechanism. The cannula insertion positions the cannula in the SQ or ID tissue through a turn of the patch pump housing relative to the device base affixed to the injection site. The injection time is determined by the compound formulation and volume, the selected cannula, and the drive mechanism. In the simplest embodiment the drive is a spring(s). Alternatively an osmotic, electromechanical or other type of drive could be used to control the injection time independent of the formulation and the tissue impedance.

A disposable infuser device for rapid subcutaneous or intradermal delivery of a drug or other medical compositions to a living being is disclosed. The infuser device comprises: a housing comprising a prefilled syringe having a cannula (e.g., a needle) coupled thereto and wherein the prefilled syringe comprises the drug or other medical composition and includes a displaceable stopper for driving the drug or other medical compositions from the syringe and through the cannula and wherein the prefilled syringe comprises a syringe axis that is parallel to the skin of the living being when the infuser device is coupled thereto; an infuser base to which the housing is coupled, and wherein the infuser base is configured to releasably secure to the skin of the living being; a cannula bending mechanism associated with the housing that, when activated, bends the cannula to be substantially perpendicular to the syringe axis while penetrating the skin of the living being; a drug delivery mechanism associated with the housing that drives the displaceable stopper to deliver the drug or other medical compositions through the cannula once the cannula has penetrated the skin of the living being; and a lever that is manually displaceable by a user of the disposable infuser device, and wherein the lever activates the cannula bending mechanism and the stopper driving mechanism via a single activation of the lever.

A method of subcutaneously or intradermally infusing a drug or other medical compounds into the skin of a living being is disclosed. The method comprises: securing a syringe, prefilled with the drug or other medical compounds, within a housing that includes a base which can be releasably secured to the skin of the living being and wherein the syringe comprises a cannula (e.g., a needle) and wherein the syringe and cannula are oriented parallel to the skin; manipulating a lever associated with the housing that bends the cannula perpendicular to the skin while subsequently causing the cannula to penetrate the skin therethrough; and initiates automatic infusion of the drug or other medical compounds from the prefilled syringe, through the cannula and the skin intothe living being by continued motion of the lever.

Various features and advantages of these inventions will become apparent to those skilled in the art from the following detailed description of the currently preferred embodiments considered with reference to the accompanying drawing figures in which like reference numerals designate like elements.

### BRIEF DESCRIPTION OF THE DRAWINGS

The foregoing summary, as well as the following detailed description of preferred embodiments of the invention, will be better understood when read in conjunction with the appended drawings. For the purpose of illustrating the invention, there are shown in the drawings embodiments which are presently preferred. It should be understood, however, that the invention is not limited to the precise arrangements and instrumentalities shown. In the drawings:

FIG. 1 is a perspective side view of a preferred embodiment of a SQ and ID infuser with an integral prefilled syringe with a staked cannula and a cannula forming mechanism as stored and shown applied to a living being's skin; (FIG. 1 shown a liner which should be removed prior to the application to the skin).

FIG. 2 is a perspective side view of a preferred embodiment of a SQ and ID infuser in FIG. 1 with the needle shield removed;

FIG. 3 is perspective side view of a preferred embodiment of a SQ and ID infuser in FIG. 1 with the needle cover removed and the lower mandrel turned into operating position;

FIG. 4 is a perspective side view of a preferred embodiment of a SQ and ID infuser in FIGS. 1, 2 and 3 with the upper mandrel/lever turned and the cannula formed for SQ or ID infusion;

FIG. 4a is a perspective distal view of the SQ and ID infuser with an integral prefilled syringe in FIG. 4;

FIG. 5 is a perspective side view of the SQ and ID infuser with an integral prefilled syringe in FIG. 4 with the cannula inserted into SQ or ID tissue;

FIG. 5a is a cross sectional side view of the SQ and ID infuser with an integral prefilled syringe in FIG. 5 at the start of an infusion;

FIG. 5b is a cross sectional side view of the SQ and ID infuser with an integral prefilled syringe in FIG. 5 at the end of the infusion;

FIG. 6 is a cross sectional side view of the SQ and ID infuser with an integral prefilled syringe in FIG. 5b with the cannula withdrawn from the tissue and shielded within the infuser;

FIG. 7 is a perspective side view of a prefilled syringe with a staked cannula and an infuser rod;

FIG. 8 is a perspective side view of a syringe in FIG. 7 with some infuser components;

FIG. 9 is a perspective side view of the assembly in FIG. 8 with the needle shield removed and the needle formed for SQ and ID insertion;

FIG. 10 is a perspective bottom view of the preferred embodiment of the SQ and ID infuser in FIG. 1;

FIG. 11a is a schematic side view, FIG. 11b is a schematic top view, and FIGS. 11c-e are cross sectional schematic views of a second preferred embodiment of a SQ and ID infuser with an integral prefilled syringe with a staked cannula and a cannula forming mechanism;

FIG. 12a is a schematic side view of the second embodiment shown adhered to the skin of the living being (same comment; the liner should be removed for the patch pump to be applied to the skin), FIG. 12b is a schematic top view, and FIGS. 12c-e are cross sectional schematic views of a second preferred embodiment of a SQ and ID infuser after the needle shield is removed, the infuser is affixed to the skin at the injection site and the cannula forming components are moved by an integral lever to the cannula;

FIG. 13a is a schematic side view, FIG. 13b is a schematic top view, and

FIGS. 13c-e are cross sectional schematic views of a second preferred embodiment of a SQ and ID infuser after the cannula is fully formed by the forming components moved further by an integral lever;

FIG. 14a is a schematic side view, FIG. 14b is a schematic top view, and FIGS. 14c-e are cross sectional schematic views of a second preferred embodiment of a SQ and ID infuser with the cannula inserted SQ or ID manually and the delivery mechanism simultaneously activated;

FIG. 15a is a schematic side view, FIG. 15b is a schematic top view, and FIGS. 15c-e are cross sectional schematic views of a second preferred embodiment of a SQ and ID infuser with the infusion completed and the syringe content fully infused;

FIG. 16a is a schematic side view, FIG. 16b is a schematic top view, and FIGS. 16c-e are cross sectional schematic views of a second preferred embodiment of a SQ and ID infuser with the infusion completed and the syringe cannula extracted from the SQ or ID tissue and permanently stored inside the device;

FIG. 17 is a schematic representation of an option of the lower and upper mandrel engagement of the patch infuser of the first embodiment;

FIG. 18a is a functional representation of the cannula deployment detent of the first embodiment during cannula forming and FIG. 18b is a functional representation of the same detent when deactivated; and

FIG. 19a is a side view functional representation representation of an option of the spring drive activation approach of the first preferred embodiment of the patch infuser and FIG. 19b is an end view of the spring drive activation approach.

### DETAILED DESCRIPTION OF THE INVENTION

Certain terminology is used in the following description for convenience only and is not limiting. The words “right” and “lower” designate directions in the drawings to which reference is made. The words “inwardly,” “outwardly,” “proximally” and “distally” refer to directions toward and away from, respectively, the geometric center or orientation of the assembly, the syringe or other related parts thereof. The words “connect,” “connectable” and “connected” mean joined or linked together, either by direct engagement or through intermediate components. Unless specifically set forth herein, the terms “a,” “an” and “the” are not limited to one element, but instead should be read as meaning “at least one.” The terminology includes the above-listed words, derivatives thereof and words of similar import.

The infusers shown in the drawings are intended for rapid subcutaneous infusion using a prefilled syringe integral with the infuser. The proposed embodiments are intended to provide an inexpensive means for delivering high volume, viscous parenteral compounds by patients. The delivery is accomplished in minutes to hours with the infuser adhered to the skin of a patient. The infusion is provided subcutaneously or intradermally and is less invasive compared to intravenous or intramuscular administration. The infuser of the present invention is a disposable device, thereby providing only a one-time use after which the entire device is discarded.

Referring to the drawings the illustrations are schematic in nature and do not provide detailed description of some elements. Some components are simplified to more effectively convey the embodiment concepts.

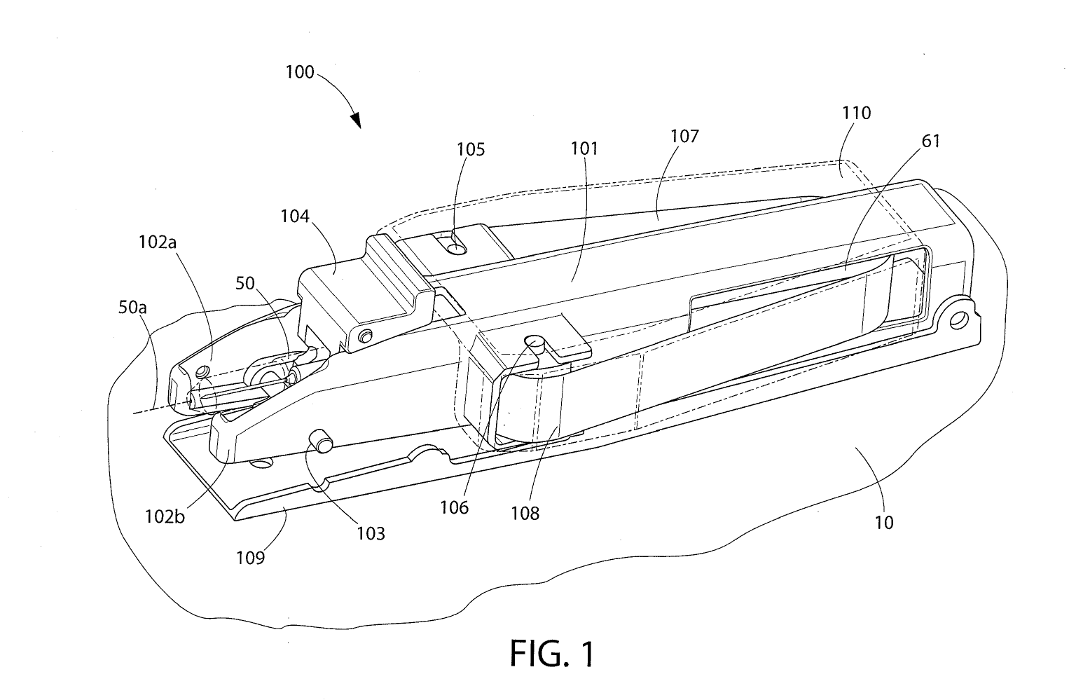

FIG. 1 illustrates a first assembly embodiment 100 whereby the prefilled syringe 50 is an integral part of the infuser 100. The infuser housing assembly 101 is comprised of a left housing 102a and a right housing 102b capturing and retaining the syringe 50. The housing is connected to a pivoting base 109. This figure also shows the device 100 applied to a living being's skin 10 and how a syringe axis 50A is aligned substantially parallel to the skin when the device 100 is first applied to the skin 10.

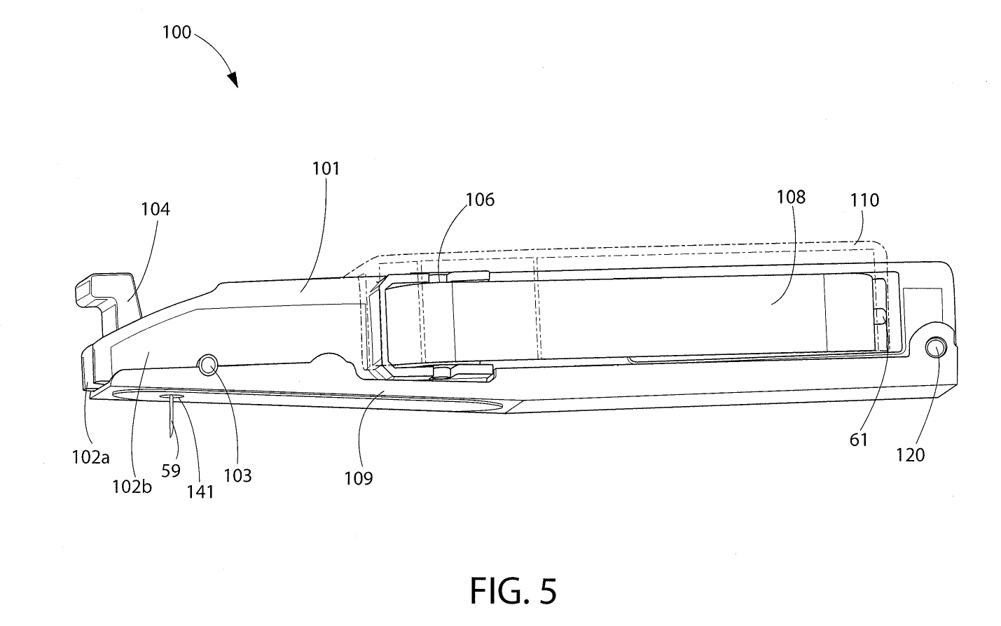

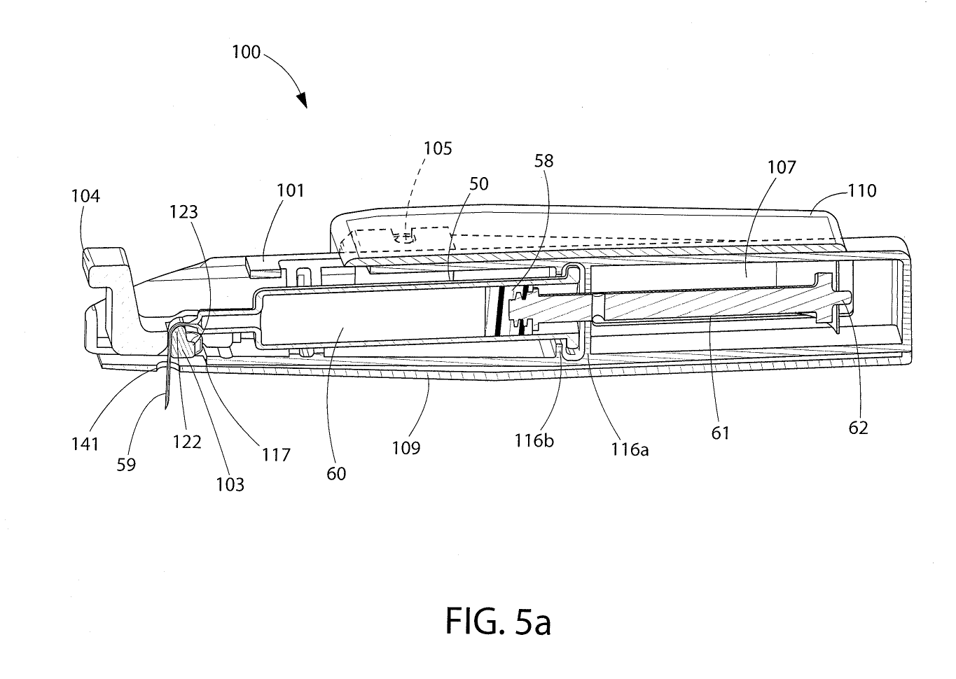

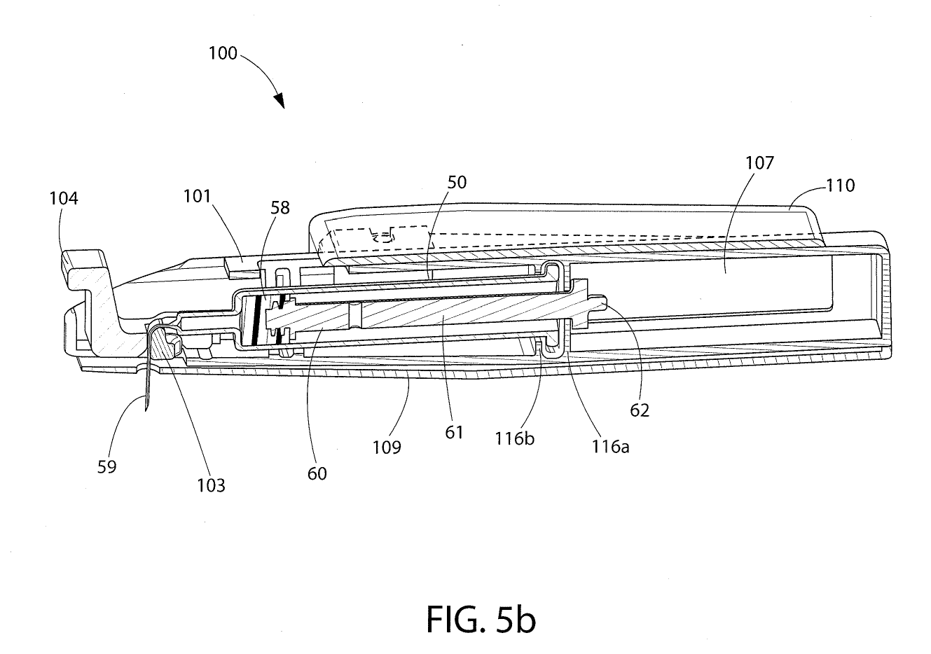

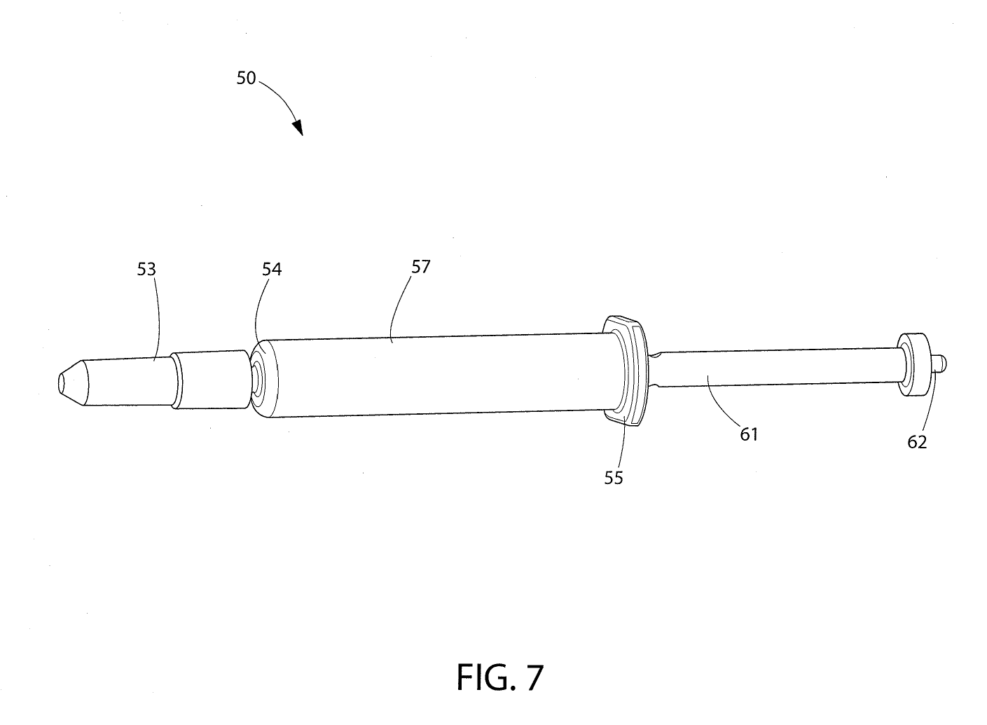

The left 102a and right 102a housings capture and retains the syringe 50 during assembly and use. The syringe 50 illustrated in FIG. 7 has a barrel 57 with a barrel distal end 54, a cannula hub 56 and a barrel flange 55. The syringe 50 is filled with a compound 60. The syringe 50 also has a stopper 58 (FIG. 5a) and a needle shield 53 closing the fluid path from the distal end. The needle shield 53 could be made from a few components and could have a rigid plastic cover. The patch injector could also have an additional component for ease of the needle shield 53 (also referred to as “needle cover”) removal. This component would be similar to that used in conventional auto injectors and is not illustrated in the figures. Furthermore the syringe has a rod 61 for the compound injection.

The injector integrates a lower mandrel 103 and an upper mandrel/lever 104. The lower mandrel 103 and the upper mandrel/lever 104 clear the needle shield 53 in their initial position. The lower mandrel 103 and the upper mandrel/lever 104 are mechanically linked by gears, cams or other mechanism. The repositioning of the mandrels is initially prevented by the needle shield of the syringe.

The compound 60 could be injected by one of the many possible drive mechanisms. These could include spring based drives, osmotic drives, electromechanical drives, etc. The preferred embodiment 100 illustrates a delivery mechanism based on constant force springs. Both constant force springs 107 and 108 are placed on bobbins 105 and 106 with the free end connected to the mount 62 of the rod 61. The spring cover 110 prevents a direct contact with the moving springs. It is shown semi-translucent to facilitate the understanding of the device design.

The syringe 50 is retained within the housing halves 102a, 102b by the protrusions 116a and 116b which capture the barrel flange 55 and via supports 119 that contact the barrel 57. The constant force springs 107 and 108 are extended and retained in storage by a mechanism preventing the distal movement of the rod 61 till it is released at the completion of the cannula insertion process. The trigger operation principle is described below.

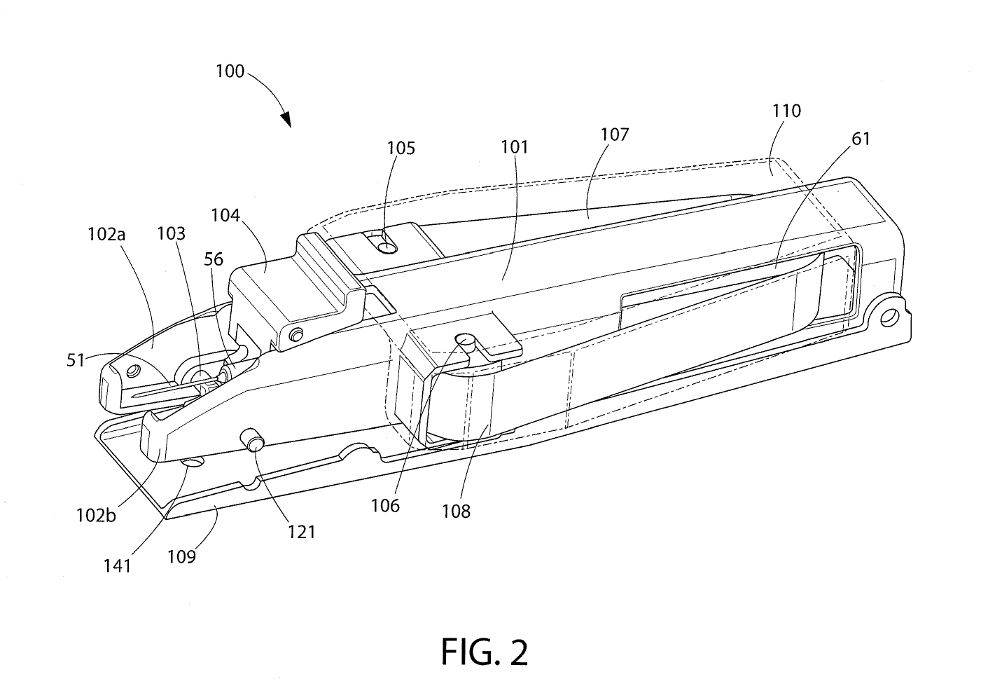

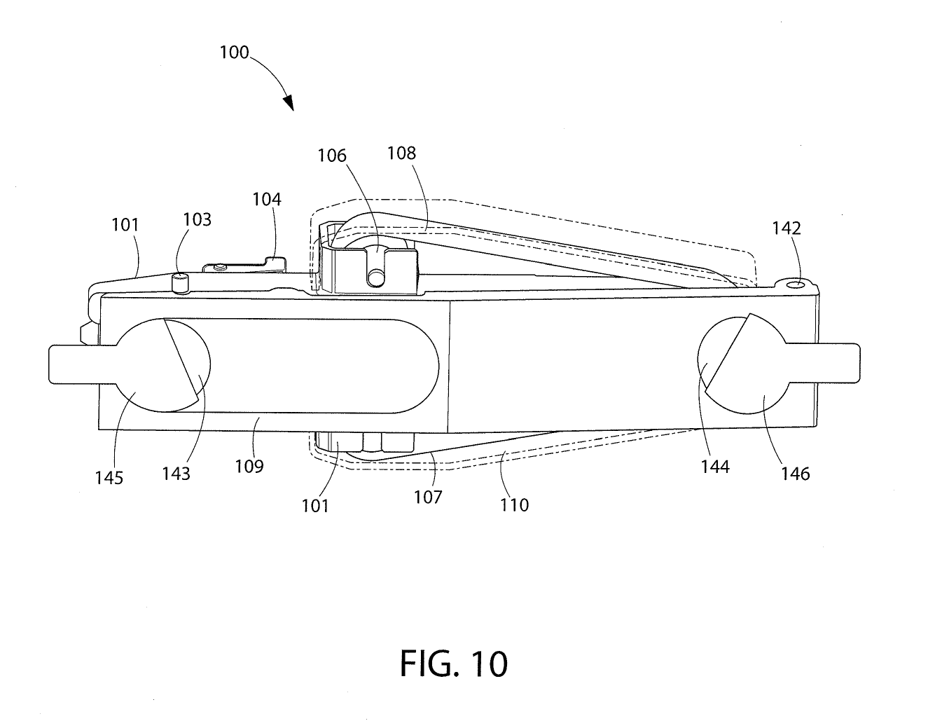

The injector base 109 has proximal openings 142 for the assembly with the base pivots 120 of the housings 102a and 102b. Furthermore the base 109 has a cannula opening 141. The injector base 109 has also adhesive elements or coatings 143 and 145 for adhering to the patient skin. The adhesive elements 143 and 145 are protected by liners 144 and 146. These liners are partially removed in FIG. 10 to expose the adhesive elements 143 and 144 underneath for clarity of construction. The adhesive elements 143 and 144 and liners could be formed as one component. The adhesive and liners are not illustrated in FIGS. 1 through 9.

The user checks the compound 60 status in the syringe by observing it through a syringe observation opening 115. The needle shield 53 is removed by the user, thereby exposing the cannula. Thereafter the liners 144 and 146 are removed from the adhesive elements 143 and 145 and the infuser is applied to an injection site. The placement of the infuser should provide the user an opportunity to visually monitor the infusion progress and to access the upper mandrel/lever for the infuser activation and removal.

The needle shield 53 of the syringe 50 prevents the repositioning of the lower and upper mandrel 104/103. The needle shield 53 could be made longer than illustrated to ease the removal of the needle shield 53 by the user. Furthermore, an additional component could be added to the needle shield 53 to make the shield removal even easier. This component would be similar to the remover assist devices used in auto injectors.

The internal components of the infuser are preferably arranged to be activated by a single lever repositioned by the user to minimize the complexity of the user activation process. In the preferred embodiment 100 the repositioning of the lever accomplishes the following actions:
1. Placing the lower mandrel 103 into operating position.
2. Placing the upper mandrel 104 into operating position.
3. Forming the needle cannula 51 (hereinafter referred to as either “needle” or “cannula” 51 and formed cannula 59) to have the distal protruding section 63 close to perpendicular orientation to the infusion site.
4. Inserting the cannula into the SQ or ID tissue.
5. Activating the compound 60 delivery mechanism.

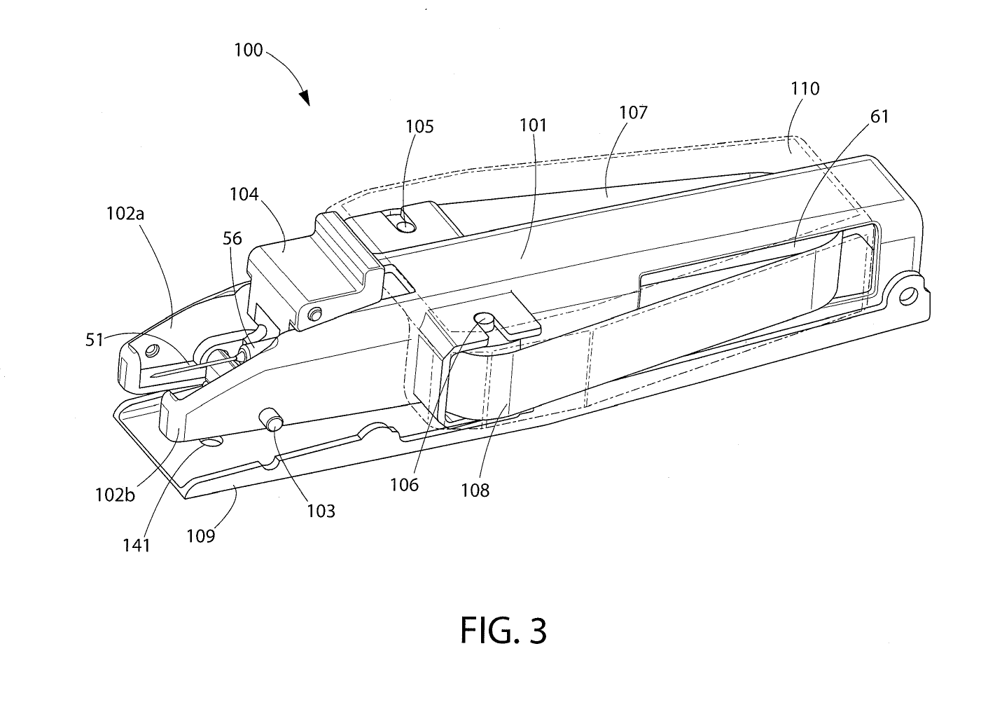

A cannula bending mechanism is described next. In particular, the upper mandrel 104 is positioned above the cannula while the lower mandrel 103 is positioned below the cannula with both the upper 104 and the lower 103 mandrels clearing the needle shield 53 in storage. In the preferred embodiment 100 the activating lever is integral with the upper mandrel. Both the upper mandrel/lever 104 and the lower mandrel 103 are repositioned by turning. The initial turn of the upper mandrel/lever 104 positions the lower mandrel close to the cannula for the forming operation as shown in FIG. 3. The mechanism engaging the lower 103 and upper mandrel/lever 104 is not illustrated.

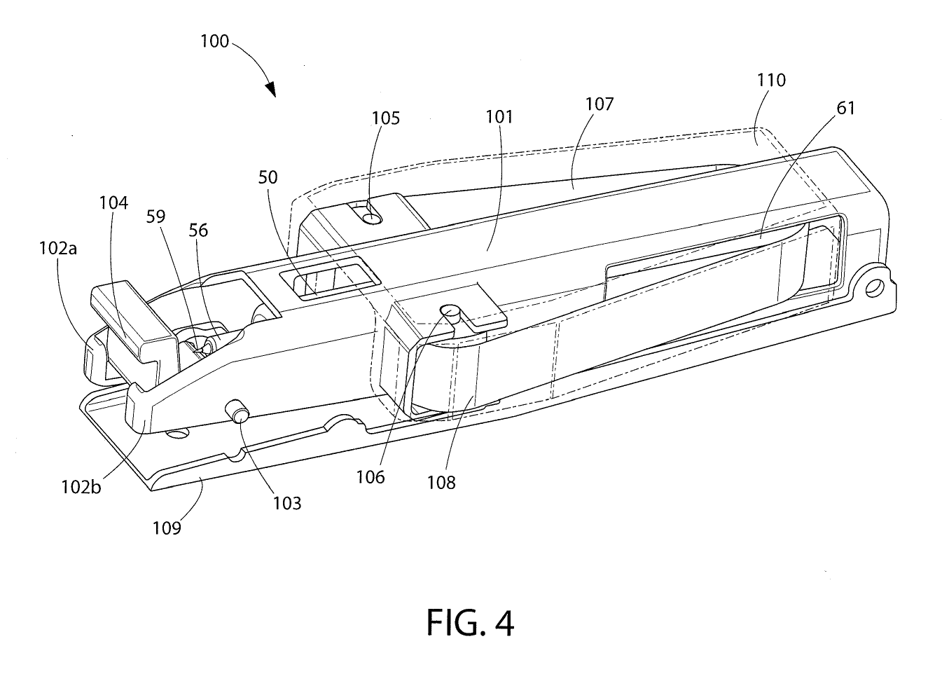

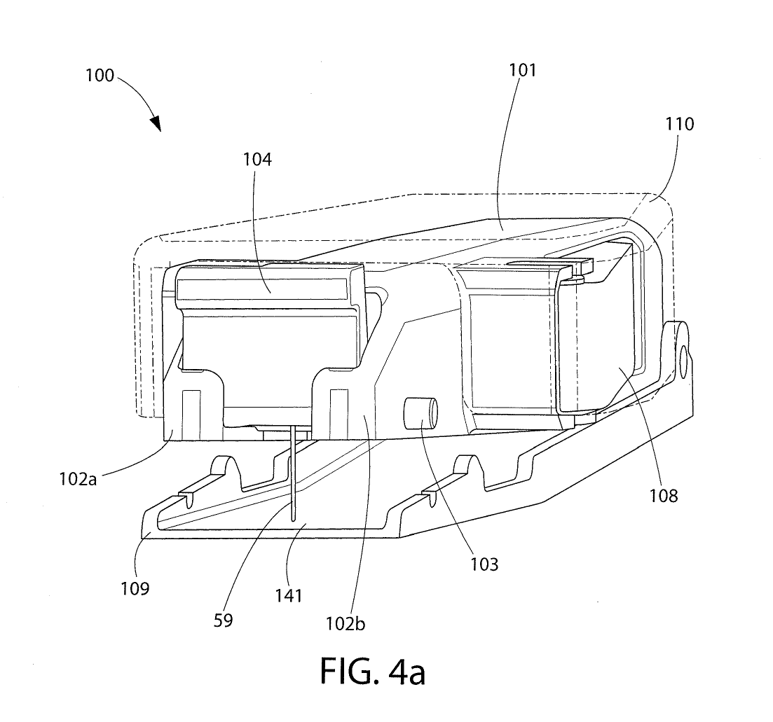

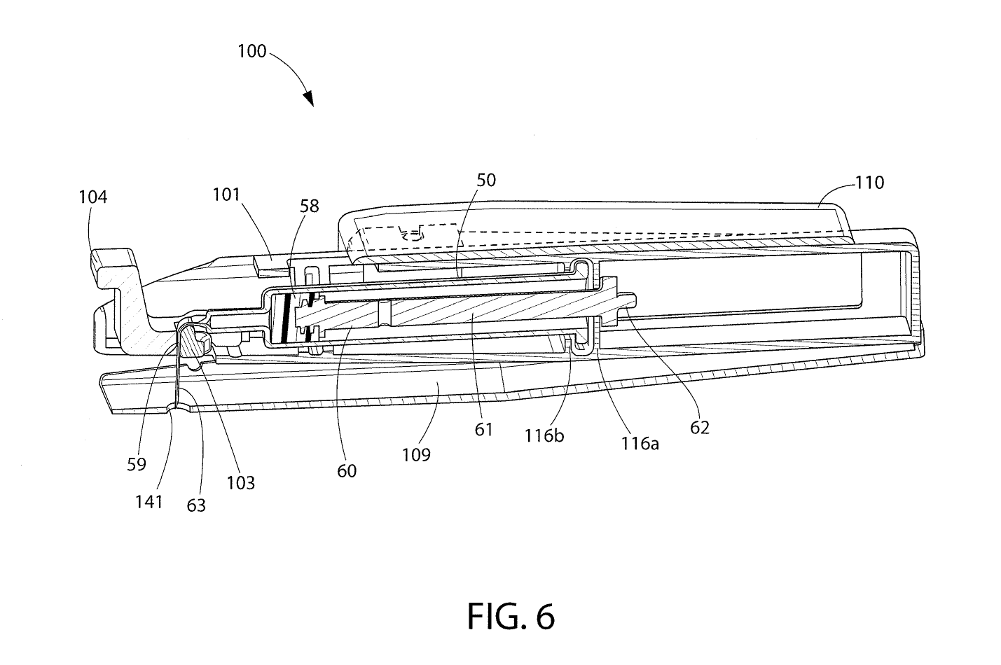

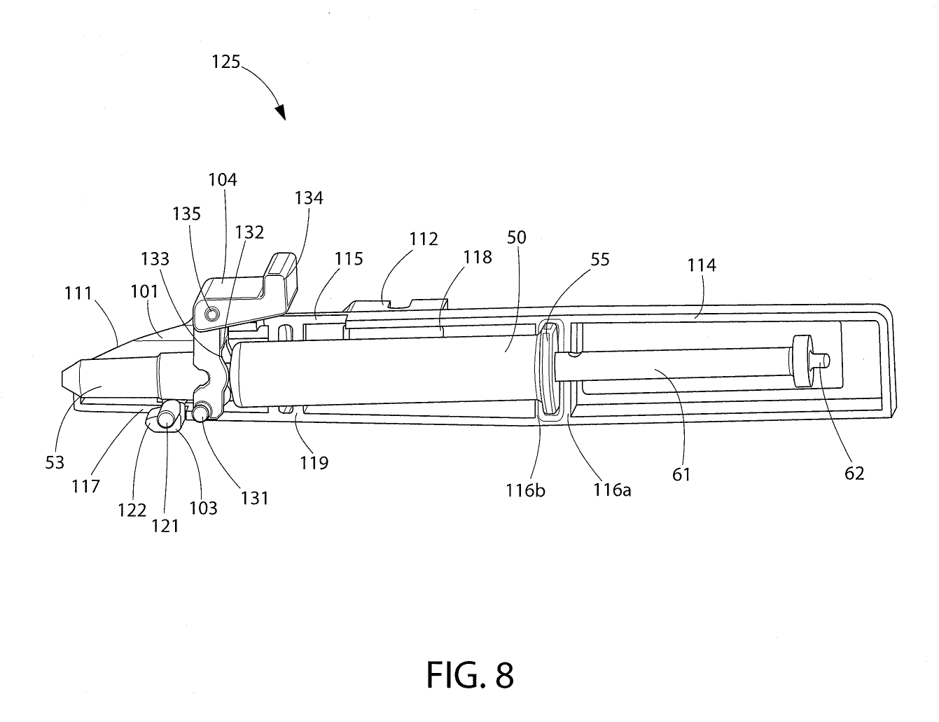

Further turning of the upper mandrel/lever 104 forms the cannula 51 as illustrated in FIG. 4. At this point the formed cannula 59 is not exposed and is hidden by the housing 101 and base 109 assembly as shown in FIG. 5. The cannula 51 is formed and locked in as formed position for insertion. The rotation of the lower mandrel 103 from its initial position to its final position can be seen most clearly by comparing FIG. 8 (initial mandrel 103 position) to FIG. 6 (final mandrel 103 position).

At this point an interlock preventing premature cannula insertion is disabled. The interlock is not illustrated. The distal section 63 of the formed cannula 59 is inserted into the tissue by turning of the housing 101 toward the base 109 as shown in FIG. 5. The formed cannula 59 is passing through a cannula opening 141 in the base 109.

Details of the infuser construction are shown FIG. 5a in a cross sectional side view. The lower mandrel 103 has a forming protrusion 122 integral with a shaft 121. The shaft is supported by openings in the housings 102a and 102b. Furthermore the lower mandrel 103 has a recess 123 for clearing the needle shield 53 during storage.

The infuser has a mechanism for the compound 60 delivery. The mechanism could be spring based, osmotic, electromechanical or of a different type. The infusion mechanism of the preferred embodiment 100 employs two constant force springs 107 and 108. The springs are placed on bobbins 105 and 106 with the free end attached to the mount 62 of the syringe rod 61. The spring bobbins 105 and 106 are mounted on spring bobbin supports 112 integral with housings 102a and 102b.

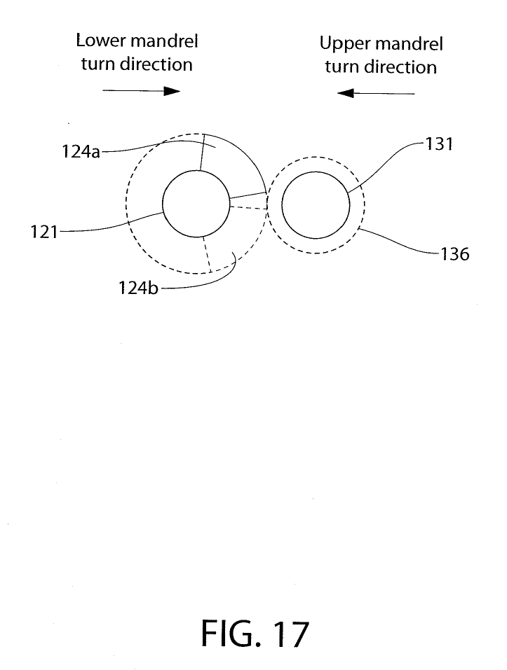

The mechanism for retaining the spring in storage and triggering the infusion are shown in FIGS. 17-19b. In particular, FIG. 17 is a schematic representation of the mandrel repositioning mechanism of the first preferred embodiment 100. The mandrels 103 and 104 are moved into operating position by turning after the needle shield 53 is removed. The lower mandrel shaft 121 is linked to the upper mandrel pivots 131 by gears. The partial gear 124a of the lower mandrel 103 is engaged to the gear 136 of the upper mandrel/lever 104. A counter clockwise turn of the upper mandrel/lever 104 moves the partial gear 124a clockwise to the position 124b at which point the lower mandrel 103 is in an operating position. The upper mandrel/lever 104 is disengaged from the lower mandrel 103 with the lower mandrel 103 retained in this operating position by a latch (not illustrated).

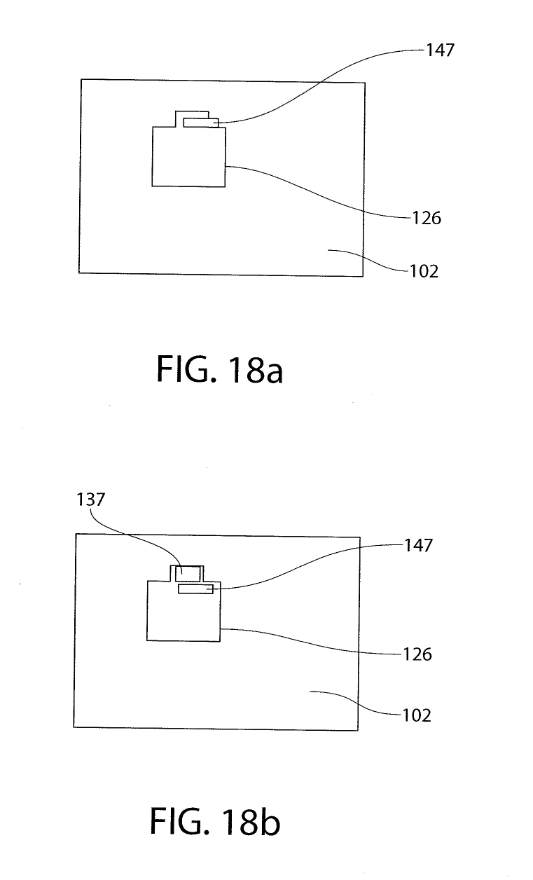

FIG. 18 is a schematic representation of the cannula deployment detent of the first embodiment 100 whereby FIG. 18a illustrates the detent during cannula forming and FIG. 18b illustrates the detent being deactivated. The housing 102 has an opening 126. The protrusion 147 of the base 109 prevents the cannula deployment/insertion into the tissue during the cannula 51 forming process. At the end for the cannula 51 forming process, a protrusion 137 of the upper mandrel/lever 104 deflects the base protrusion 147 and deactivates the detent as illustrated in FIG. 18b. The formed cannula 59 is inserted into the tissue by a further turn of the upper mandrel/lever 104.

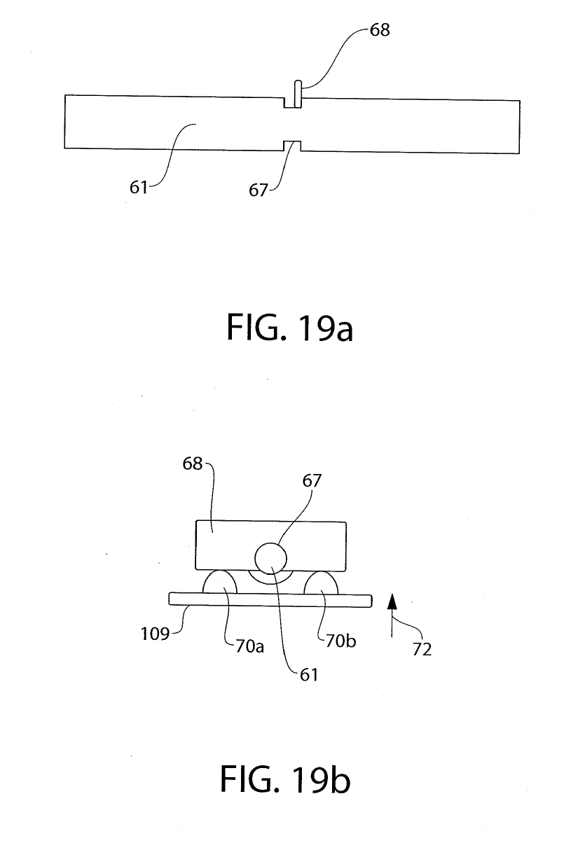

A concept of a spring drive trigger for the drug delivery mechanism is illustrated in FIG. 19. The rod 61 has an undercut 67 engaged with the spring drive retainer 68. FIG. 19a illustrates a cross section along the rod axis while FIG. 19b shows a cross section through the retainer 68. The retainer 68 is retained in its storage position by an engagement to the device housing 101. The retainer 68 prevents the rod 61 displacement. Toward the end of the insertion of the cannula 59 into the tissue by the device lever 104, protrusions 70A/70B from the base 109 act on the retainer 68 in the direction illustrated by arrow 72 in FIG. 19b. The drive retainer 68 is disengaged from the rod undercut 67 and the compound 60 delivery is initiated by the spring drive.

The compound delivery mechanism is triggered at the end of the cannula insertion illustrated in FIG. 5a. The compound 60 is infused till the full syringe content is delivered as shown in FIG. 5b. The infusion time will be determined by the combination of infusion volume, compound viscosity, cannula gage and length, and drive parameters. The compound volume could be 0.5 to 3 ml or other determined by the used prefilled syringe. It is expected that the compound could be infused in less than an hour. Any contact of the user and the user clothing with the springs is prevented prior and during infusion by the spring cover 110 attached to the housing.

The infusion progress could be monitored through the observation opening 115. The end of the compound 60 delivery is visually confirmed by the user by the stopper 58 position visible through the syringe observation opening 115. Alternatively the end of delivery could be based on the expected maximum delivery time. At the end of delivery the housing with the syringe is pulled up by the user to hide the cannula inside the injector as illustrated in FIG. 6. The cannula retraction into the device could be done by pulling on the protruding section of the upper mandrel/lever 104 or another element of the housing assembly. An internal mechanism permanently locks the cannula distal section 63 in as shielded position for safe discard. The mechanism enabling a single insertion and retraction only is not illustrated.

After the distal cannula section is extracted from the tissue and is hidden inside the device the base 109 is unpeeled from the infusion site. The device is safe to discard.

The sterility requirements of the infuser 100 are similar to the conventional auto-injectors. The only design difference is an adhesive and a somewhat longer contact of the adhesive and the skin. The compound and the cannula are sterile. The syringe cannula 51 is contacted by the mandrels 103 and 104 during the cannula forming. The distal section 63 of the cannula 59 is inserted into the tissue. It remains sterile when formed by positioning of the mandrels 103 and 104 proximally in respect to the cannula section 63.

The use of the infuser employing a conventional prefilled syringe offers the opportunity for a low cost device suitable for viscous and high volume compound delivery. Conventional prefilled syringes are used as a primary drug closure. The cannula is formed for SQ or ID injection by the user during the device activation. The device integrating the prefilled syringe provides a form factor suitable for SQ or ID infusion at a cost close to the cost of a conventional auto-injector.

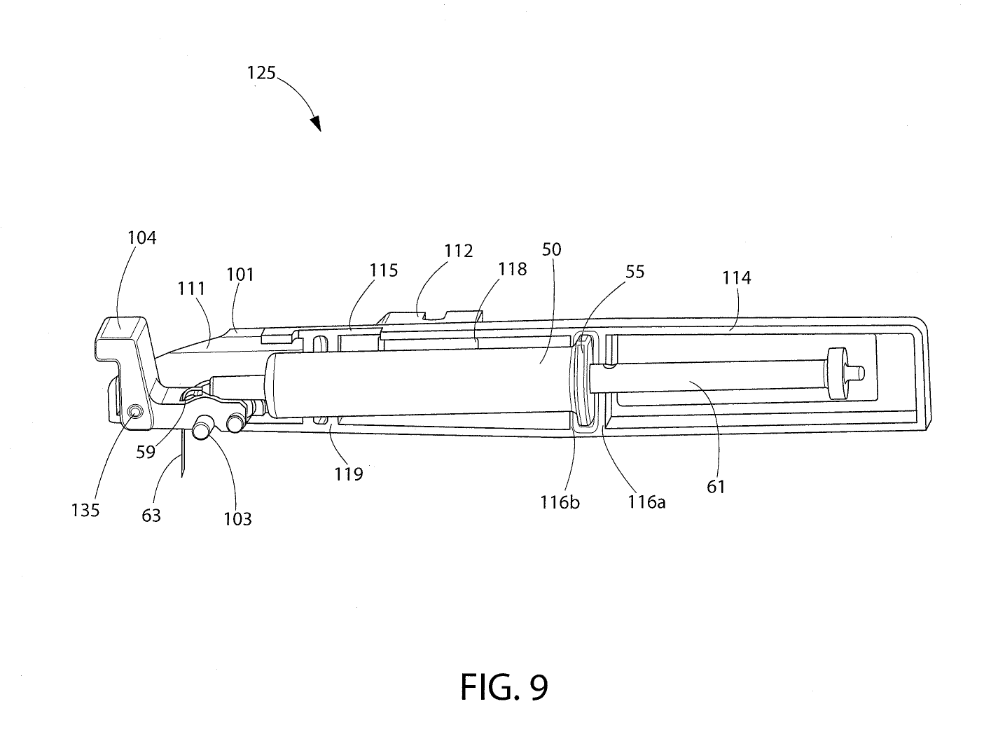

Some of the infuser design features are clarified in FIGS. 8 and 9. FIG. 8 shows a perspective side view of the infuser 100 with only some infuser components 125 while FIG. 9 is a perspective side view of the assembly in FIG. 8 with the needle shield 53 removed and the needle formed for SQ or ID insertion. The housings 102a and 102b have an opening 111 providing an access to the needle shield 53. Furthermore housings 102a and 102b have openings 117 for clearing the lower mandrel 103 in storage.

The integral upper mandrel/lever 104 of the infuser embodiment 100 has pivots 131 matching recesses of the left 102a and right 102b housings. The upper mandrel/lever has a U shaped section 133 providing clearance for the needle shield 53 in storage. The U shaped section 133 has a cannula forming surface 132 contacting and forming the cannula 51 during the turning of the upper mandrel/lever 104. The upper mandrel/lever 104 also has an integral extension 134 providing user access for the activation of the device. Protrusions 135 of the upper mandrel/lever 104 engage matching recesses in the housing halves 102a and 102b and maintain the upper mandrel/lever 104 position after activation.

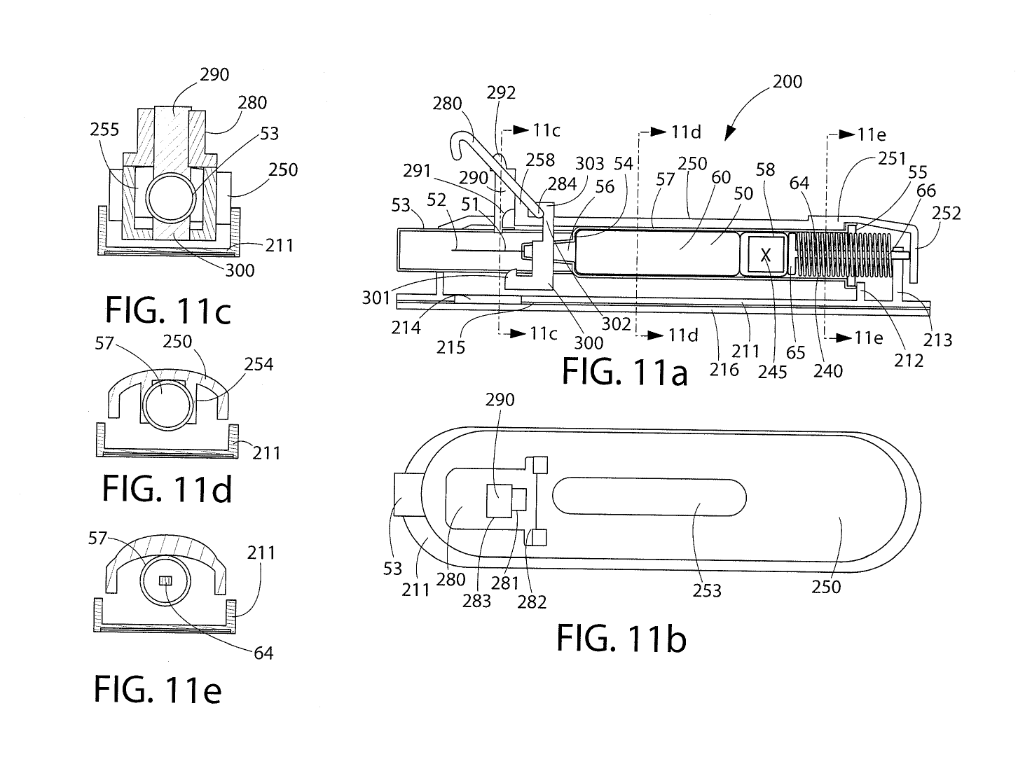

Another embodiment of a SQ and ID infuser integrating a prefilled syringe is illustrated in FIGS. 11 through 16. FIG. 11 illustrates an assembly embodiment 200 whereby the prefilled syringe 50 is an integral part of the infuser 200. The infuser 200 has a base 211 and a housing 250. The base 211 has an adhesive layer 215 for affixing the infuser to the patient skin. The adhesive 215 is protected by a protective liner 216. The base 211 has also an opening 214 to facilitate the cannula penetration into the skin.

The syringe 50 is attached to the housing 250 by integral or separate barrel supports 254. The syringe 50 is able to move only together with the housing 250. The features of the housing 250 providing the syringe 50 retention are illustrated only partially. The side walls of the housing 250 and base 211 are also not illustrated to minimize any interference with a clear explanation of SQ infuser functionality.

The housing 250 retains the syringe 50. The syringe 50 has a cannula 51 with a distal tip 52. The syringe 50 has a barrel 57 with a barrel distal end 54, a cannula hub 56 and a barrel flange 55. The syringe 50 is filled with a compound 60. The syringe 50 also has a stopper 58 and a needle shield 53 closing the fluid path from the distal end. The needle shield 53 could be made from a few components and could have a rigid plastic cover.

The syringe 50 position is defined by a flange retaining protrusion 212 of the base 211 and by a flange retainer 251 of the housing 250. The coil infusion spring 240 is retained in storage compressed with the proximal side of the delivery rod 66 of the rod assembly 64 engaging the protrusion 213 of the lower housing 211. Furthermore the spring 240 is retained compressed by the contact with a rod flat 65 of the rod assembly 64 on the distal side. The rod assembly 64 has an opening on the proximal side of the rod 65 (not shown in Figures). The opening of the rod 66 and the retainer 213 of the lower housing 211 are engaged retaining the spring compressed. The design of the spring 240 provides a spring force with a limited force reduction during delivery. The retaining protrusions 212, 213, 251 and 252 are formed not to interfere with the spring 240 operation (retainers 251 and 212) or to engage only the rod 66 (retainer 213) or spring 240 (retainer/wall 252).

The needle shield 53 of the syringe 50 is protruding through a distal opening in the housing 250. The needle shield 53 could be made longer than illustrated to ease the removal of the needle cover by the user. It could also be shaped like a mushroom head to make the removal even easier.

The housing 250 is assembled with an activation lever 280. The lever 280 is pivoting on the housing protrusion 258. The infuser has two integral mandrels 290 and 300 for forming the cannula. The support beams 302 of the lower mandrel 300 protrude through the housing 250. The support beams 302 have lifting protrusions 303. The lifting protrusions 303 engage the distal end 284 of the lever 280. The lower mandrel has also a forming section 301 positioned under the cannula. Furthermore the lower mandrel 300 is retained in alignment by bearing extensions 256 integral with the housing 250.

The upper mandrel 290 is positioned above the cannula with both upper 290 and lower 300 mandrels clearing the needle shield 53 in storage. The upper mandrel 290 is engaged to the cannula.

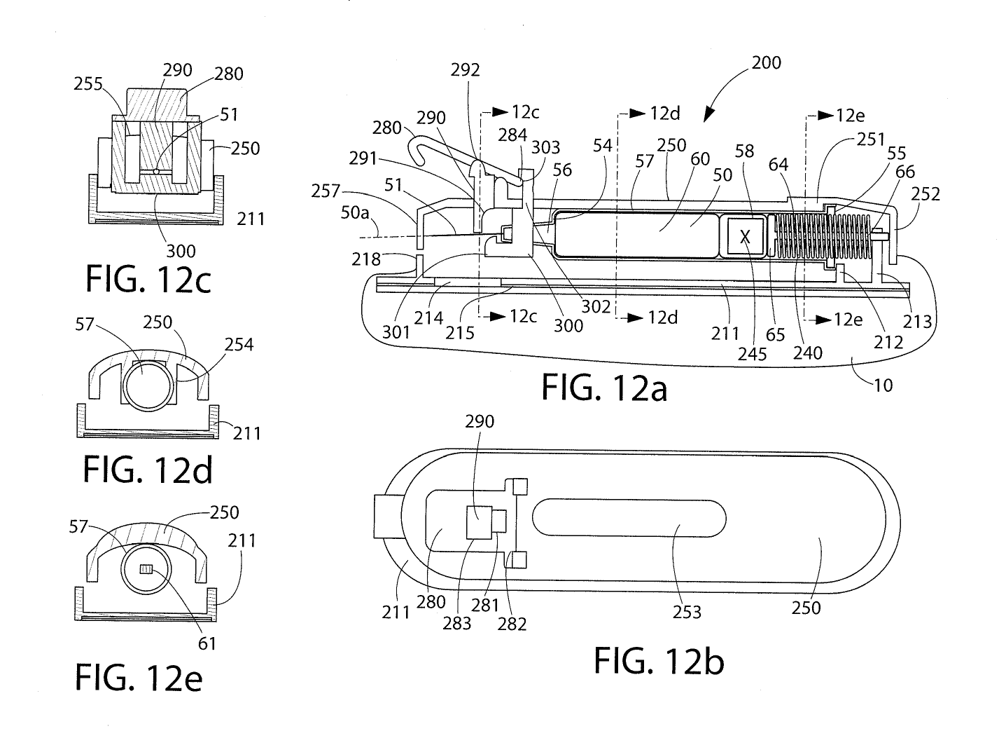

The user removes the liner 216 from the adhesive layer 215 and affixes the infuser to the skin 10 at the injection site with the syringe axis 50a in general parallel to the skin 10. There could be a number of interlocks preventing premature device activation as is commonly used in auto-injectors. The infuser 200 activation starts by turning the lever 280 pivoted on the housing 250. FIG. 12 illustrates the infuser embodiment with the cannula shield 53 removed by the user and the lever 280 turned till the upper 290 and lower 300 mandrels are moved toward the cannula with the cannula formers 291 and 301 positioned close to the cannula surface.

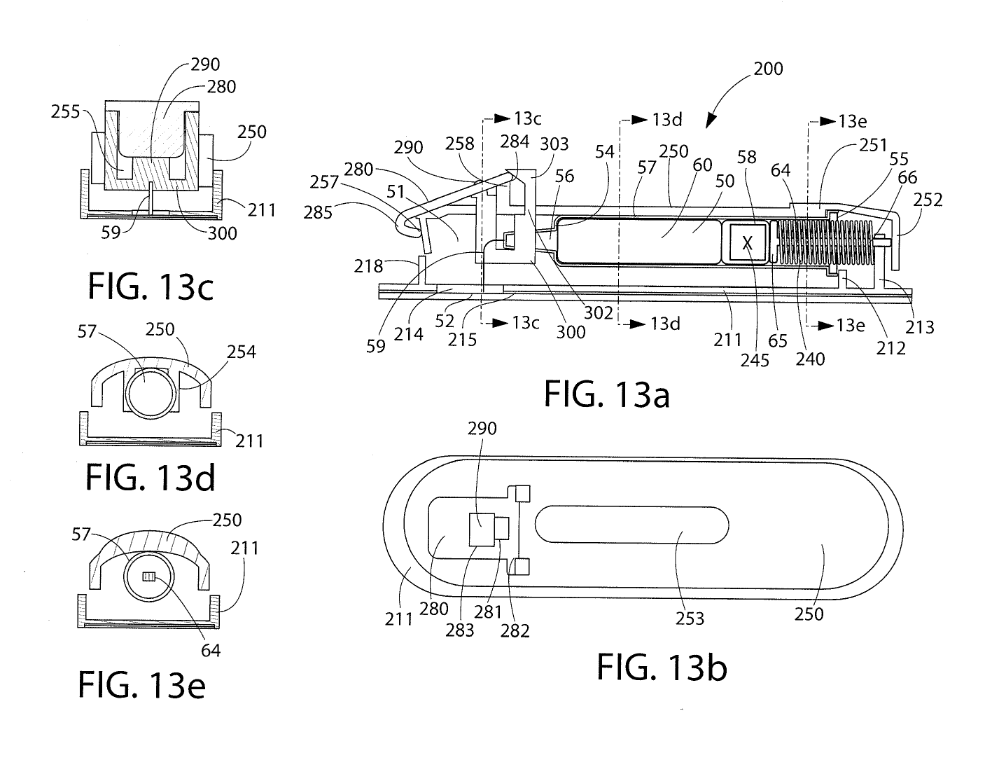

User continues to move the upper mandrel 290 down by further turning the lever 280 while the lower mandrel 300 practically does not move. This is achieved by the lever 280 engaging the lower mandrel 300 through a contact of the proximal end 284 with the lifting protrusion 303 while the lower protrusion 290 is engaged to the lever 280 by a pivot. FIGS. 13a-e illustrate the SQ infuser with the cannula 51 formed by mandrels 290 and 300.

Another feature of the infuser is an interlock preventing premature cannula insertion. Distal side walls of the housing 250 and the base 211 have contacting sections 257 and 218 positioned off center to clear the needle shield 53 (see FIG. 12a). The section 285 of the lever 280 deflects the side wall section 257 of the housing 250 as illustrated in FIG. 13a when the lever 280 is brought into contact with the housing 250. The interlock is disabled.

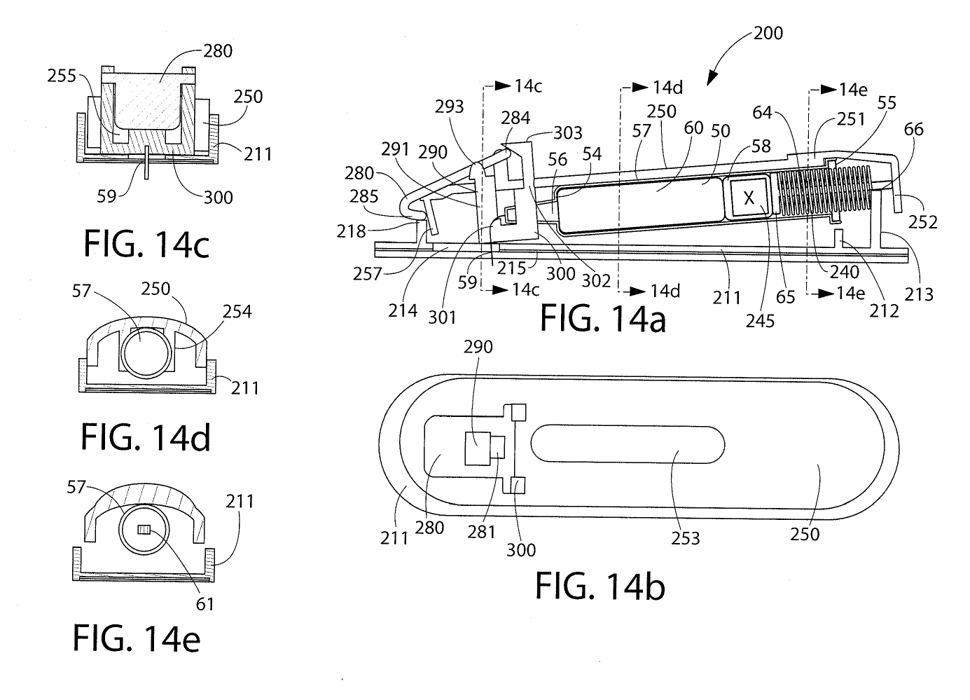

The user continues turning the lever 280 while inserting the cannula into the tissue by turning the housing 250 toward the base 211. The housing 250 and the base 211 pivot axis is marked 245. The turn of the housing 250 releases the rod 66 of the rod assembly 64 from the retainer 213 as shown in FIGS. 14a-e by lifting the proximal end of the assembly. The driving spring 240 is activated. Simultaneously the turn of the housing 250 completes the cannula 59 insertion into the tissue.

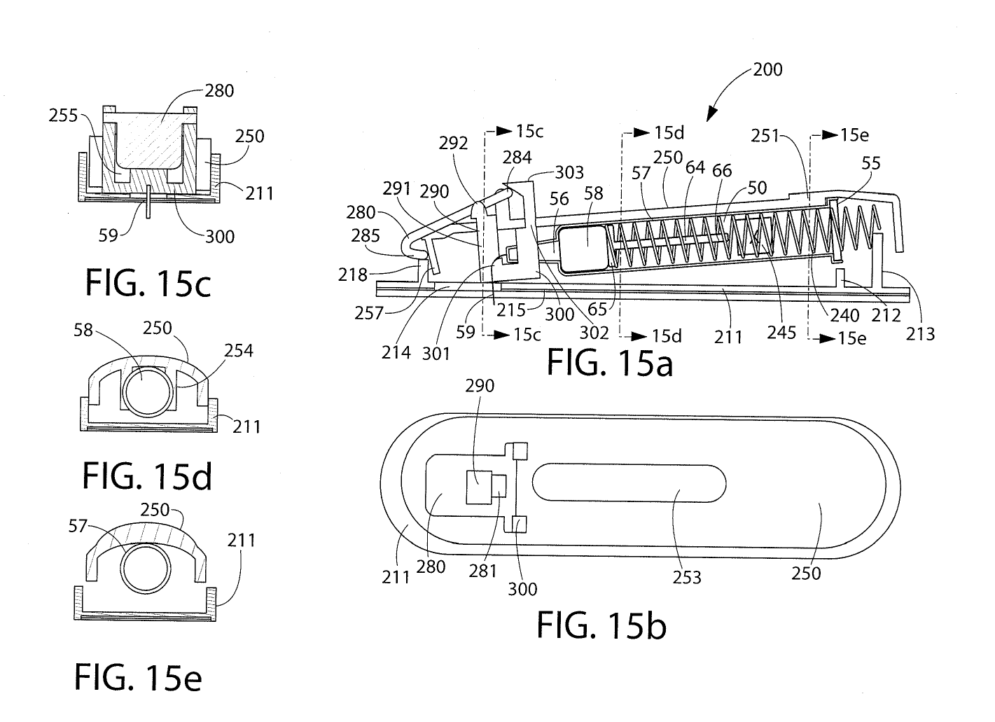

The compound delivery is initiated. The injection continues till the syringe contents is fully delivered as shown in FIGS. 15a-e. The infusion time will be determined by the combination of the infusion volume, the compound viscosity, the cannula gage and length, and the drive parameters. The compound volume could be 0.5 to 3 ml or other determined by the used prefilled syringe. It is expected that the compound could be infused in less than an hour.

The infusion progress could be monitored through the observation window 253 of the housing 250. The completion of the infusion is verified through the window 253 by the stopper 58 position.

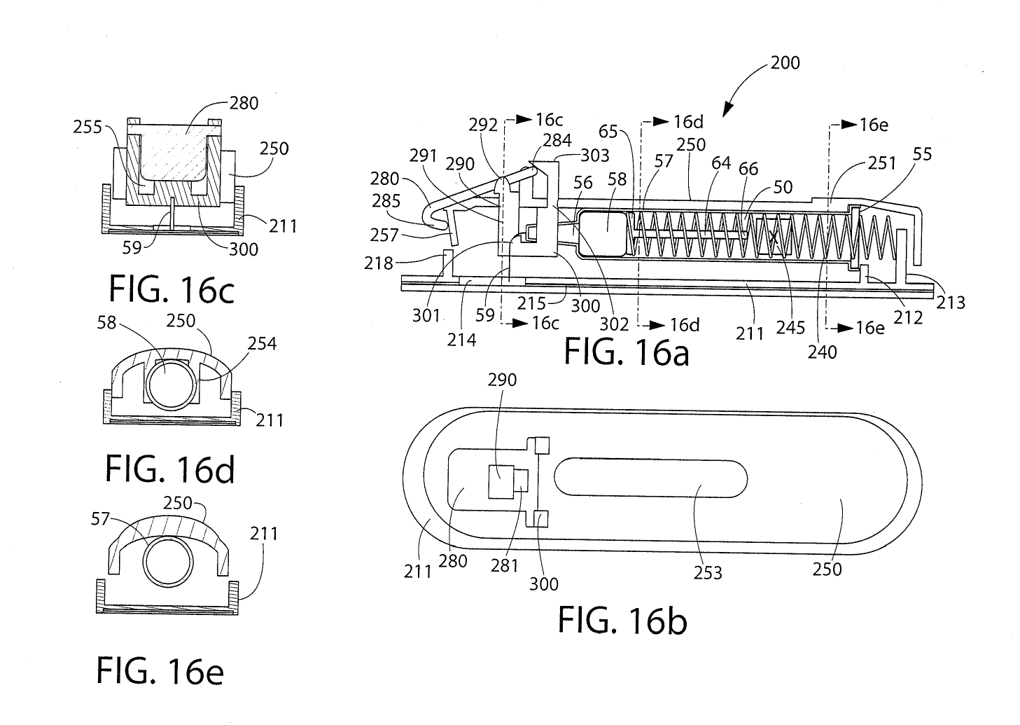

The cannula is withdrawn from the tissue by pulling on the lever 280. An interlock mechanism prevents the cannula re-exposure (not illustrated). The infuser illustrated in FIGS. 16a-e is peeled off the skin and discarded.

The base comprising a patient contact surface and securable to the patient is illustrated as planar in the second preferred embodiment 200 and as concave in the first preferred embodiment 100 while maximizing the cannula exposure length at a minimal device envelope. Alternatively the base could be convex to ideally match the potential infusion sites.

The syringe cannula 51 is conventionally made of stainless steel. The cannula 51 is glued, insert molded or secured by other means to the syringe barrel 57. Experiments showed that the cannula 51 could be bent to have the distal cannula section 63 positioned in general perpendicular to the cannula initial axis while the cannula lumen remains open. The open lumen requires the cannula 51 bending radius not to be below a minimal value determined by the cannula gage and the wall thickness. The position of the bending start should be spaced away from the cannula hub 56 to prevent any damage to the syringe barrel 57.

The cannula 51 is formed to be substantially perpendicular to the syringe 50 axis. Depending on the syringe orientation in respect to the base and other design variables the cannula 51 could be formed at an angle different from 90 degrees to the syringe 50 axis. The angle should be selected to minimize the potential distortion of the subcutaneous tissue and the skin during the cannula insertion.

It should be noted that prefilled glass syringes are characterized by substantial length tolerances. These tolerances could affect the desired lengths of the bent cannula 59 protruding through the base 109. This is in particular important when a short cannula is required. As such, the proximal and distal syringe flange retainers 116a and 116b may be adjustable along the barrel axis to accommodate these substantial length tolerances. Similarly, with regard to the second infuser embodiment, the flange retainer 251 and the distal flange retainer 212 may be adjustable along the axis of the barrel to also accommodate these substantial length tolerances of glass syringes. On the other hand, the protruding cannula length can be tightly controlled by the use of molded olefin syringes with staked cannula integrated by insert molding or other means. These “plastic” syringes have a tight tolerance of the barrel dimensions based on the employed molding process It should be further noted that installation of the syringe within the housing and any adjustments to the retainers 116a/116b or 251/212 (if needed) is accomplished at an assembly location and is not conducted by the user of the infuser devices.

Another approach is to position the syringe 50 in the housing assembly 101 with a desired position of the barrel end 54. The housing 101 features 116 capturing the syringe barrel would be spaced to enable the syringe 50 positioning in the housing accounting for the barrel 57 length tolerances. After placement of the syringe 50 the housing halves 101a and 101b the syringe 50 is permanently captured assuring the desired position of the end of barrel 57 thereby negating the impact of the tolerance of the barrel 57 length on the length of the protruding cannula section.

The patch infuser embodiments detailed above can deliver the compound subcutaneously as well as intradermally when the protruding cannula is short. The cannula protruding 0.5 to 3.5 mm past the base of the infuser embodiments infuses the compound intradermally. The rate of infusion is controlled to achieve the desired delivery rate while preventing back flow. As mentioned earlier, such control can be achieved by the cannula length/diameter and/or spring constant.

While the present invention has been described with reference to the specific embodiments thereof, it should be understood by those skilled in the art that various changes may be made and equivalents may be substituted without departing from the true spirit and scope of the invention. In addition, many modifications may be made to adapt a particular situation, material, composition of matter, method, method step or steps, for use in practicing the present invention. All such modifications are intended to be within the scope of the claims appended hereto.

All of the publications, patent applications and patents cited in this application are herein incorporated by reference in their entirety to the same extent as if each individual publication, patent application or patent was specifically and individually indicated to be incorporated by reference in its entirety.

1. A disposable infuser device for rapid subcutaneous or intradermal delivery of a drug or other medical compositions to a living being, said infuser device comprising:
a housing comprising a prefilled syringe having a cannula coupled thereto, said prefilled syringe comprising the drug or other medical composition and including a displaceable stopper for driving the drug or other medical compositions from said syringe and through said cannula and wherein said prefilled syringe comprises a syringe axis that is parallel to the skin of the living being when said infuser device is coupled thereto;
an infuser base to which said housing is coupled, said infuser base configured to releasably secure to the skin of the living being;
a cannula bending mechanism associated with said housing that, when activated, bends said cannula to be substantially perpendicular to said syringe axis while penetrating the skin of the living being;
a cannula insertion mechanism associated with said lever, said housing and said base that when activated, inserts said cannula through the skin into the underlying tissue;
a drug delivery mechanism associated with said housing and said lever that drives said displaceable stopper to deliver the drug or other medical compositions through said cannula once said cannula has penetrated the skin of the living being; and
a lever that is manually displaceable by a user of said disposable infuser device, said lever activating said cannula bending mechanism, said cannula insertion mechanism, and said stopper driving mechanism via a single activation of said lever.

2. The disposable infuser device of claim 1 wherein said cannula bending mechanism comprises first and second mandrels that are operatively connected for bending said cannula substantially perpendicular to said syringe axis.

3. The disposable infuser device of claim 2 wherein said cannula bending mechanism comprises first and second mandrels that are operatively rotatatable for bending said cannula substantially perpendicular to said syringe axis.

4. The disposable infuser device of claim 2 wherein said cannula bending mechanism comprises first and second mandrels and wherein said mandrels displace linearly for bending said cannula substantially perpendicular to said syringe axis.

5. The disposable infuser device of claim 1 wherein said drug delivery mechanism comprises constant force springs that are operatively connected to a displaceable rod that is coupled to said displaceable stopper.

6. The disposable infuser device of claim 1 wherein said drug delivery mechanism comprises compression springs that are operatively connected to a displaceable rod that is coupled to said displaceable stopper.

7. The disposable infuser device of claim 3 wherein said first mandrel comprises said lever.

8. The disposable infuser device of claim 1 further comprising an observation window that permits a user to view contents of said syringe.

9. The disposable infuser device of claim 1 further comprising an adhesive on said infuser base that releasably secures said device to the skin of the living being.

10. The disposable infuser device of claim 1 wherein said housing and said infuser base are pivotally connected.

11. The disposable infuser device of claim 10 wherein said cannula is confined between said housing and said infuser base when said drug delivery is completed and said device is removed from the skin of the living being.

12. The disposable infuser device of claim 1 wherein said housing comprises adjustable retainers to accommodate glass syringes having different length tolerances while minimizing any change in a length of said cannula that penetrates the skin.

13. The disposable infuser device of claim 1 wherein said syringe is a molded polymer syringe which minimizes any variation in a length of cannula after bending.

14. The disposable infuser device of claim 1 wherein the cannula projects past the base 0.5 to 3.5 mm to infuse the compound primarily intradermally.

15. The disposable infuser device of claim 1 wherein the cannula projects past the base over 3.5 mm to infuse the compound primarily subcutaneously.

16. A method of subcutaneously or intradermally infusing a drug or other medical compounds into the skin of a living being, said method comprising:
securing a syringe, prefilled with the drug or other medical compounds, within a housing of a device that includes a base which can be releasably secured to the skin of the living being and wherein the syringe comprises a cannula and wherein the syringe and cannula are oriented parallel to the skin;
manipulating a lever associated with said housing that bends the cannula perpendicular to the skin, causes the cannula to penetrate the skin therethrough and triggers the infusion of the drug or other medical compounds from the prefilled syringe, through the cannula and the skin of the living being by continued motion of said lever.

17. The method of claim 16 wherein said step of manipulating a lever comprises a pair of counter-rotating mandrels that bend said cannula perpendicularly for insertion into the skin.

18. The method of claim 16 wherein said step of manipulating a lever comprises a pair of mandrels which displace linearly towards the cannula and bend said cannula perpendicularly for insertion into the skin.

19. The method of claim 16 wherein said step of manipulating a lever comprises deactivation of housing to base interlock and insertion of the cannula through the skin.

20. The method of claim 16 wherein said step of manipulating a lever releases a spring drive retainer that permits constant force springs within said housing to drive a displaceable stopper within said prefilled syringe for delivering the drug or other medical compounds through said bent cannula.

21. The method of claim 16 wherein said step of manipulating a lever releases a compression spring that drives a displaceable stopper within said prefilled syringe for delivering the drug or other medical compounds through said bent cannula.

22. The method of claim 16 wherein said step of securing a syringe comprises pivotally coupling said housing and said base and wherein said method further comprises confining said bent cannula between said housing and said base when said drug delivery is completed and said device is removed from the skin of the living being.

23. The method of claim 16 wherein said step of securing a syringe comprises removing a cannula cover before manipulating said lever.

24. The method of claim 16 further comprising the step of visually confirming the contents of said syringe, before drug delivery, and prior to removing said device from the skin.

25. The method of claim 16 wherein said base comprises an adhesive covered by a liner sheet and wherein said step of securing a syringe comprises removing said liner and applying said adhesive against the skin of the living being.

26. The method of claim 16 wherein said housing comprises syringe retainers that are adjustable and wherein said step of securing a syringe comprises adjusting said retainers to accommodate glass syringes of different length tolerances while minimizing any change in a length of said cannula that penetrates the skin.

27. The method of claim 16 wherein said step of securing a syringe comprises securing a molded polymer syringe within said housing for minimizing any variation in a length of said cannula after bending.

28. The method of claim 16 wherein said step of causing said cannula to penetrate the skin infuses the drug or other medical compound primarily intradermally when said cannula penetrates the skin between 1 to 3.5 mm from a base portion of said housing.

29. The method of claim 16 wherein said step of causing said cannula to penetrate the skin infuses the drug or other medical compound primarily subcutaneously when said cannula penetrates the skin over 3.5 mm from a base portion of said housing.
